(19) United States
(12) Patent Application Publication
(10) Pub. No.: US 2023/0156174 A1
(43) Pub. Date: May 18, 2023

(54) SURGICAL VISUALIZATION IMAGE ENHANCEMENT

(71) Applicant: 3Dintegrated ApS, Copenhagen N (DK)

(72) Inventors: Marco D. F. Kristensen, San Francisco, CA (US); Sebastian H. N. Jensen, Copenhagen S (DK); Mathias B. Stokholm, Soborg (DK); Job Van Dieten, Rodovre (DK); Johan M. V. Bruun, Hellerup (DK); Steen M. Hansen, Holte (DK)

(21) Appl. No.: 17/528,369

(22) Filed: Nov. 17, 2021

(51) Int. Cl. H04N 13/282; A61B 1/00; A61B 1/04; A61B 1/018; G06T 15/20

(52) U.S. Cl. CPC H04N 13/282; A61B 1/00045; A61B 1/04; A61B 1/018; A61B 1/00154; G06T 15/20; A61B 1/313

(57) ABSTRACT

A surgical visualization system may capture images of an interior of a cavity of a patient with a plurality of cameras. Those images may subsequently be used to create a three dimensional point cloud representing the interior of the cavity of the patient. This point cloud may then be used as a basis for displaying a representation of the interior of the cavity of the patient, which representation may be manipulated or viewed from different perspectives without necessarily requiring movement of any physical camera.

### BACKGROUND

Surgical systems may incorporate an imaging system, which may allow the clinician(s) to view the surgical site and/or one or more portions thereof on one or more displays such as a monitor. The display(s) may be local and/or remote to a surgical theater. An imaging system may include a scope with a camera that views the surgical site and transmits the view to a display that is viewable by the clinician. Scopes include, but are not limited to, laparoscopes, robotic laparoscopes, arthroscopes, angioscopes, bronchoscopes, choledochoscopes, colonoscopes, cytoscopes, duodenoscopes, enteroscopes, esophagogastro-duodenoscopes (gastroscopes), endoscopes, laryngoscopes, nasopharyngo-neproscopes, sigmoidoscopes, thoracoscopes, ureteroscopes, and exoscopes. Imaging systems may be limited by the information that they are able to recognize and/or convey to the clinician(s). For example, limitations of cameras used in capturing images may result in reduced image quality.

Examples of surgical imaging systems are disclosed in U.S. Pat. Pub. No. 2020/0015925, entitled “Combination Emitter and Camera Assembly,” published Jan. 16, 2020; U.S. Pat. Pub. No. 2020/0015923, entitled “Surgical Visualization Platform,” published Jan. 16, 2020; U.S. Pat. Pub. No. 2020/0015900, entitled “Controlling an Emitter Assembly Pulse Sequence,” published Jan. 16, 2020; U.S. Pat. Pub. No. 2020/0015899, entitled “Surgical Visualization with Proximity Tracking Features,” published Jan. 16, 2020; U.S. Pat. Pub. No. 2020/0015924, entitled “Robotic Light Projection Tools,” published Jan. 16, 2020; and U.S. Pat. Pub. No. 2020/0015898, entitled “Surgical Visualization Feedback System,” published Jan. 16, 2020. The disclosure of each of the above-cited U.S. patents and patent applications is incorporated by reference herein.

While various kinds of surgical instruments and systems have been made and used, it is believed that no one prior to the inventor(s) has made or used the invention described in the appended claims.

### BRIEF DESCRIPTION OF THE DRAWINGS

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1 depicts a schematic view of an exemplary surgical visualization system including an imaging device and a surgical device;

FIG. 2 depicts a schematic diagram of an exemplary control system that may be used with the surgical visualization system of FIG. 1;

FIG. 3 depicts image processing which may be applied to images prior to display for a surgeon;

FIG. 4 depicts a method for processing images using learned image signal processing;

FIG. 5 depicts a scenario in which a plurality of imaging devices are used to gather data for an exemplary surgical visualization system;

FIGS. 6A-6B depict relationships between images captured with a single imaging device and multiple imaging devices; and

FIG. 7 depicts a method which may be performed to provide visualizations based on data captured by a plurality of imaging devices.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical device. The term “proximal” refers to the position of an element arranged closer to the surgeon, and the term “distal” refers to the position of an element arranged further away from the surgeon. Moreover, to the extent that spatial terms such as “top,” “bottom,” “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

Furthermore, the terms “about,” “approximately,” and the like as used herein in connection with any numerical values or ranges of values are intended to encompass the exact value(s) referenced as well as a suitable tolerance that enables the referenced feature or combination of features to function for the intended purpose(s) described herein.

Similarly, the phrase “based on” should be understood as referring to a relationship in which one thing is determined at least in part by what it is specified as being “based on.” This includes, but is not limited to, relationships where one thing is exclusively determined by another, which relationships may be referred to using the phrase “exclusively based on.”

I. Exemplary Surgical Visualization System

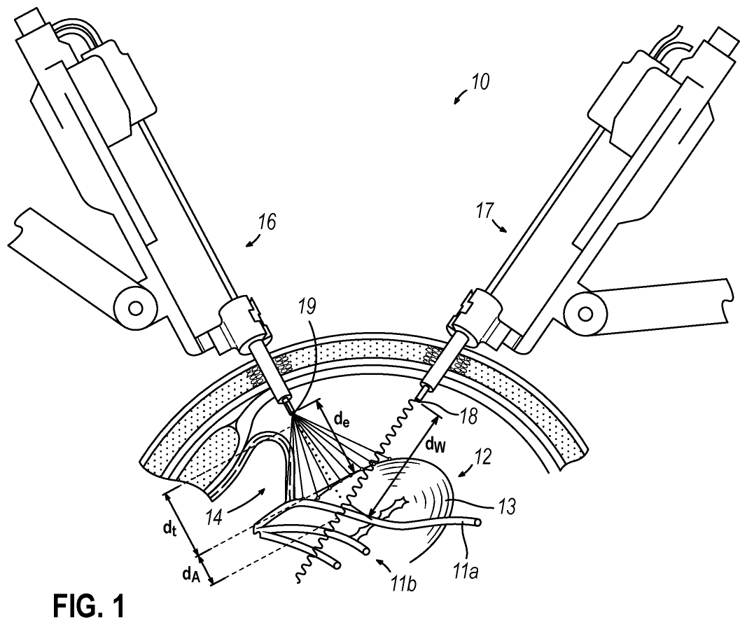

FIG. 1 depicts a schematic view of a surgical visualization system (10) according to at least one aspect of the present disclosure. The surgical visualization system (10) may create a visual representation of a critical structure (11a, 11b) within an anatomical field. The surgical visualization system (10) may be used for clinical analysis and/or medical intervention, for example. In certain instances, the surgical visualization system (10) may be used intraoperatively to provide real-time, or near real-time, information to the clinician regarding proximity data, dimensions, and/or distances during a surgical procedure. The surgical visualization system (10) is configured for intraoperative identification of critical structure(s) and/or to facilitate the avoidance of critical structure(s) (11a, 11b) by a surgical device. For example, by identifying critical structures (11a, 11b), a clinician may avoid maneuvering a surgical device into a critical structure (11a, 11b) and/or a region in a predefined proximity of a critical structure (11a, 11b) during a surgical procedure. The clinician may avoid dissection of and/or near a vein, artery, nerve, and/or vessel, for example, identified as a critical structure (11a, 11b), for example. In various instances, critical structure(s) (11a, 11b) may be determined on a patient-by-patient and/or a procedure-by-procedure basis.

Critical structures (11a, 11b) may be any anatomical structures of interest. For example, a critical structure (11a, 11b) may be a ureter, an artery such as a superior mesenteric artery, a vein such as a portal vein, a nerve such as a phrenic nerve, and/or a sub-surface tumor or cyst, among other anatomical structures. In other instances, a critical structure (11a, 11b) may be any foreign structure in the anatomical field, such as a surgical device, surgical fastener, clip, tack, bougie, band, and/or plate, for example. In one aspect, a critical structure (11a, 11b) may be embedded in tissue. Stated differently, a critical structure (11a, 11b) may be positioned below a surface of the tissue. In such instances, the tissue conceals the critical structure (11a, 11b) from the clinician's view. A critical structure (11a, 11b) may also be obscured from the view of an imaging device by the tissue. The tissue may be fat, connective tissue, adhesions, and/or organs, for example. In other instances, a critical structure (11a, 11b) may be partially obscured from view. A surgical visualization system (10) is shown being utilized intraoperatively to identify and facilitate avoidance of certain critical structures, such as a ureter (11a) and vessels (11b) in an organ (12) (the uterus in this example), that are not visible on a surface (13) of the organ (12).

### A. Overview of Exemplary Surgical Visualization System

With continuing reference to FIG. 1, the surgical visualization system (10) incorporates tissue identification and geometric surface mapping, potentially in combination with a distance sensor system (14). In combination, these features of the surgical visualization system (10) may determine a position of a critical structure (11a, 11b) within the anatomical field and/or the proximity of a surgical device (16) to the surface (13) of the visible tissue and/or to a critical structure (11a, 11b). The surgical device (16) may include an end effector having opposing jaws (not shown) and/or other structures extending from the distal end of the shaft of the surgical device (16). The surgical device (16) may be any suitable surgical device such as, for example, a dissector, a stapler, a grasper, a clip applier, a monopolar RF electrosurgical instrument, a bipolar RF electrosurgical instrument, and/or an ultrasonic instrument. As described herein, a surgical visualization system (10) may be configured to achieve identification of one or more critical structures (11a, 11b) and/or the proximity of a surgical device (16) to critical structure(s) (11a, 11b).

The depicted surgical visualization system (10) includes an imaging system that includes an imaging device (17), such as a camera or a scope, for example, that is configured to provide real-time views of the surgical site. In various instances, an imaging device (17) includes a spectral camera (e.g., a hyperspectral camera, multispectral camera, a fluorescence detecting camera, or selective spectral camera), which is configured to detect reflected or emitted spectral waveforms and generate a spectral cube of images based on the molecular response to the different wavelengths. Views from the imaging device (17) may be provided to a clinician; and, in various aspects of the present disclosure, may be augmented with additional information based on the tissue identification, landscape mapping, and input from a distance sensor system (14). In such instances, a surgical visualization system (10) includes a plurality of subsystems—an imaging subsystem, a surface mapping subsystem, a tissue identification subsystem, and/or a distance determining subsystem. These subsystems may cooperate to intraoperatively provide advanced data synthesis and integrated information to the clinician(s).

The imaging device (17) of the present example includes an emitter (18), which is configured to emit spectral light in a plurality of wavelengths to obtain a spectral image of hidden structures, for example. The imaging device (17) may also include a three-dimensional camera and associated electronic processing circuits in various instances. In one aspect, the emitter (18) is an optical waveform emitter that is configured to emit electromagnetic radiation (e.g., near-infrared radiation (NIR) photons) that may penetrate the surface (13) of a tissue (12) and reach critical structure(s) (11a, 11b). The imaging device (17) and optical waveform emitter (18) thereon may be positionable by a robotic arm or a surgeon manually operating the imaging device. A corresponding waveform sensor (e.g., an image sensor, spectrometer, or vibrational sensor, etc.) on the imaging device (17) may be configured to detect the effect of the electromagnetic radiation received by the waveform sensor.

The wavelengths of the electromagnetic radiation emitted by the optical waveform emitter (18) may be configured to enable the identification of the type of anatomical and/or physical structure, such as critical structure(s) (11a, 11b). The identification of critical structure(s) (11a, 11b) may be accomplished through spectral analysis, photo-acoustics, fluorescence detection, and/or ultrasound, for example. In one aspect, the wavelengths of the electromagnetic radiation may be variable. The waveform sensor and optical waveform emitter (18) may be inclusive of a multispectral imaging system and/or a selective spectral imaging system, for example. In other instances, the waveform sensor and optical waveform emitter (18) may be inclusive of a photoacoustic imaging system, for example. In other instances, an optical waveform emitter (18) may be positioned on a separate surgical device from the imaging device (17). By way of example only, the imaging device (17) may provide hyperspectral imaging in accordance with at least some of the teachings of U.S. Pat. No. 9,274,047, entitled “System and Method for Gross Anatomic Pathology Using Hyperspectral Imaging,” issued Mar. 1, 2016, the disclosure of which is incorporated by reference herein in its entirety.

The depicted surgical visualization system (10) also includes an emitter (19), which is configured to emit a pattern of light, such as stripes, grid lines, and/or dots, to enable the determination of the topography or landscape of a surface (13). For example, projected light arrays may be used for three-dimensional scanning and registration on a surface (13). The projected light arrays may be emitted from an emitter (19) located on a surgical device (16) and/or an imaging device (17), for example. In one aspect, the projected light array is employed to determine the shape defined by the surface (13) of the tissue (12) and/or the motion of the surface (13) intraoperatively. An imaging device (17) is configured to detect the projected light arrays reflected from the surface (13) to determine the topography of the surface (13) and various distances with respect to the surface (13). By way of further example only, a visualization system (10) may utilize patterned light in accordance with at least some of the teachings of U.S. Pat. Pub. No. 2017/0055819, entitled “Set Comprising a Surgical Instrument,” published Mar. 2, 2017, the disclosure of which is incorporated by reference herein in its entirety; and/or U.S. Pat. Pub. No. 2017/0251900, entitled “Depiction System,” published Sep. 7, 2017, the disclosure of which is incorporated by reference herein in its entirety.

The depicted surgical visualization system (10) also includes a distance sensor system (14) configured to determine one or more distances at the surgical site. In one aspect, the distance sensor system (14) may include a time-of-flight distance sensor system that includes an emitter, such as the structured light emitter (19); and a receiver (not shown), which may be positioned on the surgical device (16). In other instances, the time-of-flight emitter may be separate from the structured light emitter. In one general aspect, the emitter portion of the time-of-flight distance sensor system (14) may include a laser source and the receiver portion of the time-of-flight distance sensor system (14) may include a matching sensor. A time-of-flight distance sensor system (14) may detect the “time of flight,” or how long the laser light emitted by the structured light emitter (19) has taken to bounce back to the sensor portion of the receiver. Use of a very narrow light source in a structured light emitter (19) may enable a distance sensor system (14) to determine the distance to the surface (13) of the tissue (12) directly in front of the distance sensor system (14).

Referring still to FIG. 1, a distance sensor system (14) may be employed to determine an emitter-to-tissue distance (dₑ) from a structured light emitter (19) to the surface (13) of the tissue (12). A device-to-tissue distance (dₜ) from the distal end of the surgical device (16) to the surface (13) of the tissue (12) may be obtainable from the known position of the emitter (19) on the shaft of the surgical device (16) relative to the distal end of the surgical device (16). In other words, when the distance between the emitter (19) and the distal end of the surgical device (16) is known, the device-to-tissue distance (dₜ) may be determined from the emitter-to-tissue distance (dₑ). In certain instances, the shaft of a surgical device (16) may include one or more articulation joints; and may be articulatable with respect to the emitter (19) and the jaws. The articulation configuration may include a multi-joint vertebrae-like structure, for example. In certain instances, a three-dimensional camera may be utilized to triangulate one or more distances to the surface (13).

As described above, a surgical visualization system (10) may be configured to determine the emitter-to-tissue distance (dₑ) from an emitter (19) on a surgical device (16) to the surface (13) of a uterus (12) via structured light. The surgical visualization system (10) is configured to extrapolate a device-to-tissue distance (dₜ) from the surgical device (16) to the surface (13) of the uterus (12) based on emitter-to-tissue distance (dₑ). The surgical visualization system (10) is also configured to determine a tissue-to-ureter distance (d_{A}) from a ureter (11a) to the surface (13) and a camera-to-ureter distance (d_{w}), from the imaging device (17) to the ureter (11a). Surgical visualization system (10) may determine the camera-to-ureter distance (d_{w}), with spectral imaging and time-of-flight sensors, for example. In various instances, a surgical visualization system (10) may determine (e.g., triangulate) a tissue-to-ureter distance (d_{A}) (or depth) based on other distances and/or the surface mapping logic described herein.

### B. Exemplary Control System

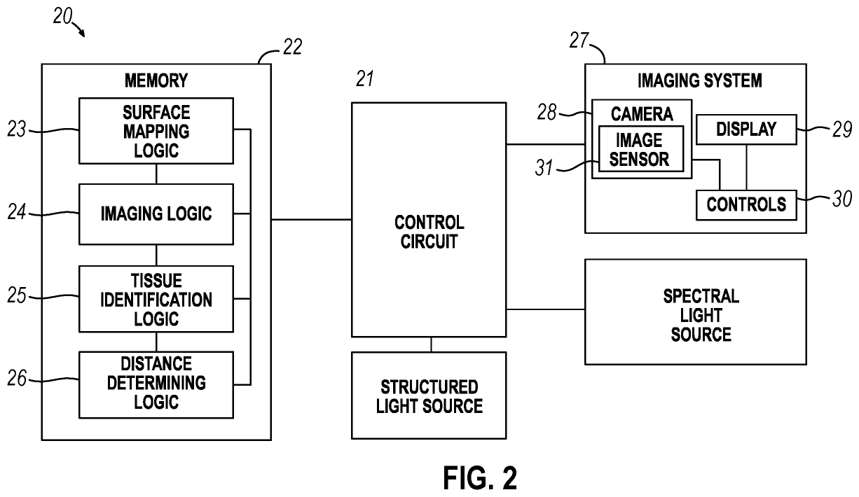

FIG. 2 is a schematic diagram of a control system (20), which may be utilized with a surgical visualization system (10). The depicted control system (20) includes a control circuit (21) in signal communication with a memory (22). The memory (22) stores instructions executable by the control circuit (21) to determine and/or recognize critical structures (e.g., critical structures (11a, 11b) depicted in FIG. 1), determine and/or compute one or more distances and/or three-dimensional digital representations, and to communicate certain information to one or more clinicians. For example, a memory (22) stores surface mapping logic (23), imaging logic (24), tissue identification logic (25), or distance determining logic (26) or any combinations of logic (23, 24, 25, 26). The control system (20) also includes an imaging system (27) having one or more cameras (28) (like the imaging device (17) depicted in FIG. 1), one or more displays (29), one or more controls (30) or any combinations of these elements. The one or more cameras (28) may include one or more image sensors (31) to receive signals from various light sources emitting light at various visible and invisible spectra (e.g., visible light, spectral imagers, three-dimensional lens, among others). The display (29) may include one or more screens or monitors for depicting real, virtual, and/or virtually-augmented images and/or information to one or more clinicians.

In various aspects, a main component of a camera (28) includes an image sensor (31). An image sensor (31) may include a Charge-Coupled Device (CCD) sensor, a Complementary Metal Oxide Semiconductor (CMOS) sensor, a short-wave infrared (SWIR) sensor, a hybrid CCD/CMOS architecture (sCMOS) sensor, and/or any other suitable kind(s) of technology. An image sensor (31) may also include any suitable number of chips.

The depicted control system (20) also includes a spectral light source (32) and a structured light source (33). In certain instances, a single source may be pulsed to emit wavelengths of light in the spectral light source (32) range and wavelengths of light in the structured light source (33) range. Alternatively, a single light source may be pulsed to provide light in the invisible spectrum (e.g., infrared spectral light) and wavelengths of light on the visible spectrum. A spectral light source (32) may include a hyperspectral light source, a multispectral light source, a fluorescence excitation light source, and/or a selective spectral light source, for example. In various instances, tissue identification logic (25) may identify critical structure(s) via data from a spectral light source (32) received by the image sensor (31) portion of a camera (28). Surface mapping logic (23) may determine the surface contours of the visible tissue based on reflected structured light. With time-of-flight measurements, distance determining logic (26) may determine one or more distance(s) to the visible tissue and/or critical structure(s) (11a, 11b). One or more outputs from surface mapping logic (23), tissue identification logic (25), and distance determining logic (26), may be provided to imaging logic (24), and combined, blended, and/or overlaid to be conveyed to a clinician via the display (29) of the imaging system (27).

II. Exemplary Surgical Visualization System with Multi-Step or Machine Learning Image Enhancement

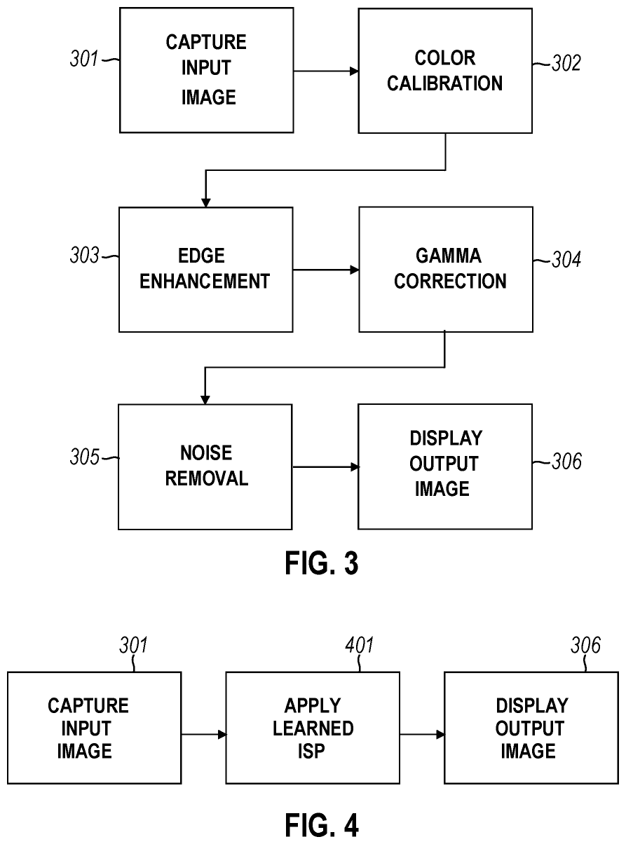

In some instances, it may be desirable to provide a surgical visualization system that is configured to use machine learning or other types of processing to circumvent limitations of equipment used in capturing data (e.g., imaging device (17)). To illustrate, consider FIG. 3, which depicts image processing which may be applied to images captured by an imaging device (17) prior to being displayed for a surgeon. In the process of FIG. 3, in step (301) an input image is captured, e.g., through the direct detection of light by imaging device (17). This image may then be color calibrated in step (302). For example, prior to capturing the image in step (301), an image of a target having known color characteristics may be captured using the imaging device (17), and the color of the target in the image captured by the imaging device (17) may be compared with the target's known color characteristics. This comparison may then be used to create a data structure, such as a filter or mask for transforming the colors in the image as captured to match the correct color characteristics of the target. Subsequently, in step (302), the color calibration may be performed by applying this data structure to the input image captured in step (301) to correct for color distortions associated with the imaging device (17).

Continuing with the discussion of FIG. 3, after color calibration in step (302), the depicted process continues with edge enhancement in step (303). This may be done, for example, by preparing a kernel that could enhance all edges in an input image, or that may enhance edges having an orientation matching an expected orientation of edges in a critical structure. This kernel may then be convolved with the input image to prepare an edge enhanced image in which edges (e.g., critical structure edges) are more easily perceived when the image is presented on a display (e.g., display (29)). Following edge enhancement in step (303), the process of FIG. 3 continues with gamma correction in step (304). In the gamma correction step (304), the image as encoded by the imaging device (17) may be translated into a display image to compensate for compression that may have been applied by the imaging device (17) itself. The image may then be subjected to noise removal in step (305). This noise removal may be performed in a manner similar to that described in the context of the edge enhancement of step (303). For example, a kernel may be created to remove noise that may have been introduced by the imaging device (17) such as through the use of a sliding window filter such as a mean or median filter, or through a custom filter created by imaging a known target using the imaging device (17) and determining transformations needed to convert the image of the known target as captured by the imaging device (17) to match the actual undistorted target. Finally, this processed image may be displayed in step (306), such as by presenting it on display (29) for use by a surgeon in performing a procedure.

Variations on a process such as shown in FIG. 3 may also be utilized in some cases. For example, in some cases additional steps beyond those illustrated in FIG. 3 may be performed, such as by applying an additional sharpening step (e.g., through unsharp masking) to further improve the image that would be displayed in step (306) relative to the image captured in step (301). Similarly, in some cases steps such as shown in FIG. 3 may be applied in a different order than indicated. For instance, in some cases, gamma correction of step (304) may be performed prior to color calibration and/or edge enhanced of steps (302) and (303), or may be performed after noise removal (305) or other processing steps (e.g., sharpening). Other variations on an image processing approach such as shown in FIG. 3 may also be performed and will be immediately apparent to one of ordinary skill based on this disclosure, and so the method of FIG. 3 should be understood as being illustrative only, and should not be treated as implying limitations on the protection provided by this document or any related document.

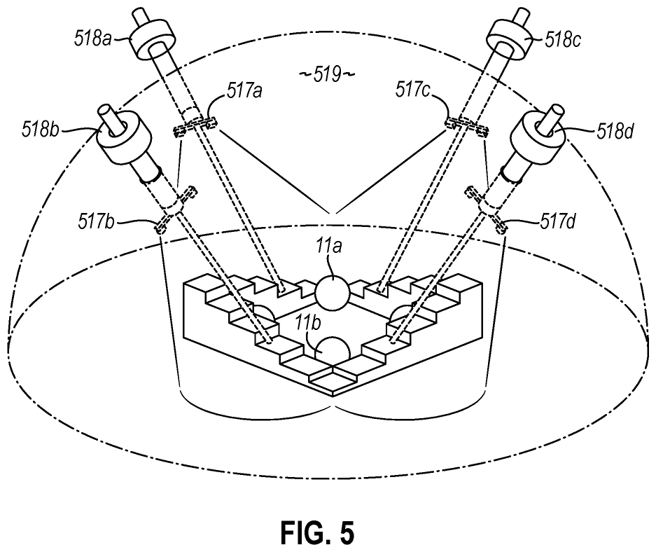

It should also be understood that multi-phase processes such as shown in FIG. 3 are not the only types of image enhancements that may be utilized in systems implemented based on this disclosure. For example, as illustrated in FIG. 4, rather than applying multiple image processing steps such as steps (302)-(305), in some cases an input image may be transformed to a display image through the application of learned image signal processing in step (401). To implement this type of learned image signal processing, in some cases, prior to capturing the input image (301), a set of training data may be captured, such as by capturing a plurality of raw images using the imaging device (17), as well as by capturing a plurality of corresponding images depicting the same scene as the raw images, but doing so in a manner that captures data that would be equivalent to the processed images that would be displayed in step (306). These corresponding images may be captured, for instance, using a larger laparoscope than would be used with imaging device (17) (thereby allowing for the corresponding images to be captured with a higher quality camera), and/or by illuminating the scene with better lighting than would be expected with the imaging device (17). The corresponding images may also (or alternatively) be images subjected to some level of image processing, such as that described in the context of FIG. 3. However, these images were obtained, once the raw and corresponding images were available, they could be used as training data to generate a machine learning model (e.g., a convolutional neural network) such as could be applied in step (401). Accordingly, just as the steps illustrated in FIG. 3 should not be treated as implying limitations on multi-step image processing methods that may be applied based on this disclosure, FIG. 4 and its accompanying description should be understood as being illustrative only, and should not be treated as limiting.

III. Exemplary Surgical Visualization System with Multi-Camera Image Combination

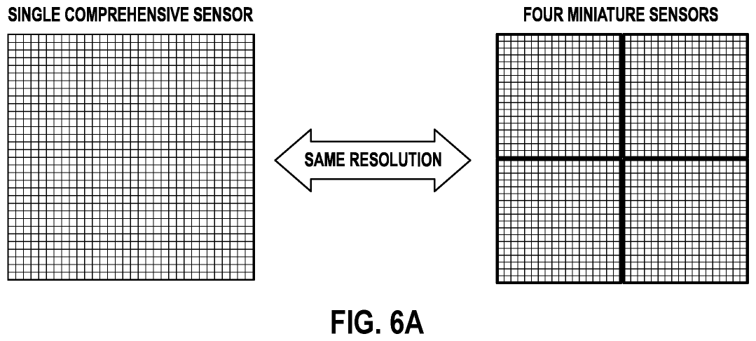

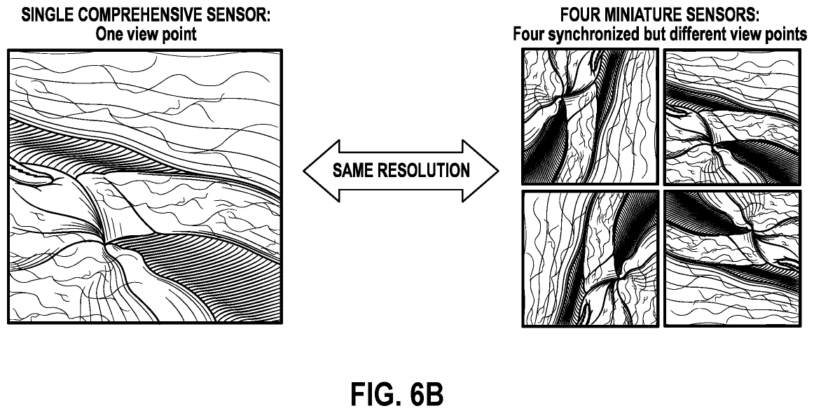

In some cases, it may be desirable to combine data captured from multiple imaging devices to provide a more robust and versatile data set. To illustrate, consider a scenario such as shown in FIG. 5. FIG. 5 illustrates a scenario in which a plurality of imaging devices (517a, 517b, 517c, 517d) are at least partially inserted through corresponding trocars (518a)(518b)(518c)(518d) to capture images of an interior of a cavity of a patient (519). As shown in FIG. 5, each of the imaging devices (517a, 517b, 517c, 517d) has a corresponding field of view, and those fields of view overlap to provide a complete view of the portion of the interior of the cavity of the patient, including one or more critical structures (11a, 11b) (represented by spheres in FIG. 5). In such a case, image processing techniques such as bundle adjustment or other multi view geometry techniques may be used to combine the images captured by the various imaging devices (517a, 517b, 517c, 517d) to create a complete three dimensional representation (e.g., a point cloud) of the relevant portion of the interior of the cavity of the patient (519). This, in turn, may allow for the imaging devices used in capturing the images to be smaller than would be the case if a single imaging device were relied on, as their combined viewpoints may allow for sufficient information to be captured despite the limitations of any individual device, as is shown in FIGS. 6A and 6B. This may allow, for example, imaging devices having a cross sectional area less than one square millimeter to be used. An example of such a device is the OV6948 offered by OmniVision Technologies, Inc., which measures 0.575 mm×0.575 mm, though the use of that particular imaging device is intended to be illustrative only, and should not be treated as implying limitations on the types of imaging devices which may be used in a scenario such as shown in FIG. 5. For example, in some cases, one or more imaging devices used to capture images of the interior of the cavity of the patient may be stereo cameras, which could have a larger cross sectional area than may be present in a non-stereo imaging device.

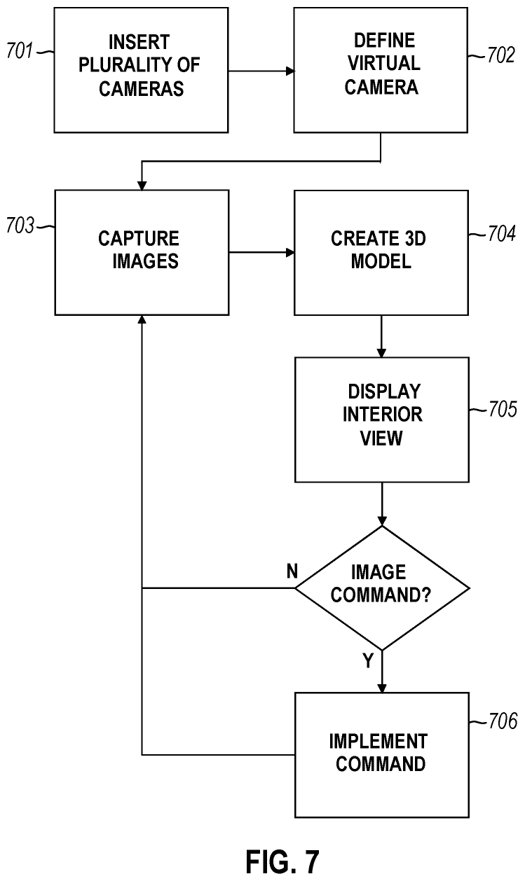

Turning now to FIG. 7, that figure illustrates a method which may be performed to provide visualizations in a multi-camera scenario such as shown in FIG. 5. Initially, in step (701), a plurality of cameras would be inserted to capture images of an interior of a cavity of a patient, such as by inserting cameras through trocars as shown in FIG. 5. Additionally, a virtual camera position would be defined in step (702). This may be done, for example, by identifying a likely location of one or more critical structures (11a, 11b) in the interior of a cavity of a patient (519), such as based on where those structure(s) were located in a CT or other pre-operative image, and placing the virtual camera at a location where it would capture those critical structure(s) in its field of view. After the virtual camera is defined in step (702), image data is captured from each of a plurality of sensors in step (703). This may be done, for example, using a plurality of imaging devices (517a, 517b, 517c, 517d) disposed such as shown in FIG. 5 to capture images of the interior of the cavity of the patient. In step (704) these images may be combined to produce a comprehensive 3D model of the interior of the patient, such as using bundle adjustment or other multi-view geometry techniques to create a point cloud representing the interior as reflected in the images captured in step (703). This 3D model may then be used in step (705) to display a view of the interior of the cavity of the patient from the viewpoint of the virtual camera.

In a method such as shown in FIG. 7, if the surgeon wished to view the interior of the cavity of the patient from a different viewpoint (e.g., to get a better view of a critical structure (11a, 11b)), he or she could provide a command indicating how the displayed image should be changed. If such a command was received then, in step (706) the command could be implemented by modifying the virtual camera, such as by changing its position, focus, or orientation, depending on the desired change in the displayed image. In some cases, this may allow the view of the interior of the cavity of the patient to be changed without requiring movement of any physical camera, though in some cases one or more physical imaging devices (517a, 517b, 517c, 517d) may also (or alternatively) be moved in order to implement the command in step (706). The process may then return to step (703) to capture additional images (e.g., so that any changes, such as movement of a critical structure during a procedure would be displayed), and may cycle through steps (703), (704), (705) and (potentially) (706) thereby providing a continuously updated real time image of the interior of the cavity of the patient until the procedure was complete.

It should be understood that FIGS. 5-7 and their associated description are intended to be illustrative only, and that variations on those methods and configurations will be immediately apparent to, and could be implemented without undue experimentation by, those of ordinary skill in the art in light of this disclosure. For example, while FIG. 5 illustrated a scenario on which four imaging devices (517a, 517b, 517c, 517d) were used to capture images of the interior of a cavity of a patient, it is possible that fewer (e.g., 2 or 3) or more (e.g., 10 or more imaging devices) may be used in certain contexts when implementing the disclosed technology. Similarly, while FIG. 7 indicated that the steps of image capture and 3D model creation would be repeatedly performed to provide real time visualizations of the interior of the cavity of a patient, in some cases one or more of these steps may be performed more intermittently. For example, in some cases a new 3D model may be created only once every five frames, while other frames may simply reskin the most recently created 3D model with then current images of the interior of the patient (potentially after performing some level of enhancement of those images, such as overlaying indications of critical structures identified using spectral processing), thereby potentially reducing processing requirements and latency for a system implemented based on this disclosure. This same type of technique may be used in some cases to allow three dimensional viewing of an interior of a cavity of a patient even when the camera(s) used to capture images do not have overlapping fields of view. For example, if one or more virtual cameras in a scenario such as illustrated in FIG. 5 moves such that its field of view no longer overlaps with the other camera(s), the image captured by that camera could be applied to the most recently created 3D image, or a new 3D image could be computed using extrapolation or interpolation based on the most recent previously created 3D image, thereby providing a full virtual camera view of the interior of the cavity of the patient even in a case where the fields of view of the cameras used to image the interior were not overlapping.

In some cases, variations may also be implemented on how information from individual cameras may be handled to create or visualize the interior of the cavity of the patient. For example, in some cases where one or more of the cameras used to image the interior of the cavity of the patient is a stereo camera, the known horizontal physical displacement between the imaging elements of the stereo camera may be used to provide a baseline to computer the actual scale of objects in the stereo camera's field of view. Similarly, in some cases, rather than (or in addition to) displaying images from the viewpoint of a virtual camera as discussed in the context of FIG. 7, in some cases when cameras are being inserted a surgeon may be provided with an image from one of the physical cameras (e.g., a stereo camera or other default camera, or a camera that may have been selected in advance of the procedure). The surgeon may then subsequently switch between physical camera images (e.g., switching from an image captured by one physical camera to another), between physical and virtual camera images, or to a hybrid interface in which a virtual camera image is displayed along with one or more physical camera images. Other variations (e.g., other approaches to providing a baseline for determining physical size of objects, such as tracking the physical position of different cameras in space, or matching images captured by cameras against pre-operatively obtained images having known size information) may also be implemented, and will be immediately apparent to those of ordinary skill in the art in light of this disclosure. Accordingly, the examples set forth herein should be understood as being illustrative only, and should not be treated as implying limitations on the scope of protection provided by this document or any related document.

IV. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A surgical visualization system comprising: (a) a plurality of trocars, each trocar comprising a working channel; (b) a plurality of cameras, wherein each camera from the plurality of cameras: (i) has a corresponding trocar from the plurality of trocars; (ii) is least partially inserted through the working channel of its corresponding trocar; and (iii) is adapted to capture images of an interior of a cavity of a patient when inserted through the working channel of its corresponding trocar; (c) a processor, wherein: (i) for each camera from the plurality of cameras: (A) the processor is in operative communication with that camera; and (B) the processor is configured to receive a set of points corresponding to an image captured by that camera; and (ii) the processor is configured to generate a three dimensional point cloud representing the interior of the cavity of the patient based on combining the sets of points received from the plurality of cameras.

### Example 2

The surgical visualization system of Example 1, wherein the plurality of cameras comprises at least four cameras.

### Example 3

The surgical visualization system of any of Examples 1-2, wherein the processor is configured to combine the sets of points received from the plurality of cameras using bundle adjustment.

### Example 4

The surgical visualization system of any of Examples 1-3, wherein the processor is configured to display a view of the interior of the cavity of the patient as viewed from a viewpoint of a virtual camera based on the three dimensional point cloud.

### Example 5

The surgical visualization system of Example 4, wherein the processor is configured to, based on receiving a command to modify the view of the interior of the cavity of the patient: (a) modifying one or more of the virtual camera's position, focus and orientation; and (b) displaying an updated view of the interior of the cavity of the patient, wherein the updated view is of the interior of the cavity of the patient as viewed by the virtual camera after the modification.

### Example 6

The surgical visualization system of Example 5, wherein the processor is configured to display the updated view of the interior of the cavity of the patient while holding each of the plurality of cameras stationary.

### Example 7

The surgical visualization system of any of Examples 1-6, wherein at least one camera from the plurality of cameras has a cross sectional area less than or equal to one square millimeter.

### Example 8

A method comprising: (a) for each of a plurality of cameras inserting that camera at least partially through a corresponding trocar; (b) using the plurality of cameras to capture images of an interior of a cavity of a patient; and (c) a processor: (i) receiving, from each camera from the plurality of cameras, a set of points corresponding to an image captured by that camera; and (ii) generating a three dimensional point cloud representing the interior of the cavity of the patient based on combining the sets of points.

### Example 9

The method of Example 8, wherein the plurality of cameras comprises at least four cameras.

### Example 10

The method of any of Examples 8-9, wherein the processor is configured to combine the sets of points received from the plurality of cameras using bundle adjustment.

### Example 11

The method of any of Examples 8-10, wherein the processor is configured to display a view of the interior of the cavity of the patient as viewed from a viewpoint of a virtual camera based on the three dimensional point cloud.

### Example 12

The method of Example 11, wherein the processor is configured to, based on receiving a command to modify the view of the interior of the cavity of the patient: (a) modify one or more of the virtual camera's position, focus and orientation; and (b) display an updated view of the interior of the cavity of the patient, wherein the updated view is of the interior of the cavity of the patient as viewed by the virtual camera after the modification.

### Example 13

The method of Example 12, wherein the processor is configured to display the updated view of the interior of the cavity of the patient while holding each of the plurality of cameras stationary.

### Example 14

The method of any of Examples 8-13, wherein each camera from the plurality of cameras has a cross sectional area less than or equal to one square millimeter.

### Example 15

A non-transitory computer readable medium storing instructions operable to configure a surgical visualization system to perform a set of steps comprising: (a) capturing, using a plurality of cameras, a plurality of images of an interior of a cavity of a patient; (b) receiving, from each camera from the plurality of cameras, a set of points corresponding to an image captured by that camera; and (c) generating a three dimensional point cloud representing the interior of the cavity of the patient based on combining the sets of points.

### Example 16

The medium of Example 15, wherein the plurality of cameras comprises four cameras.

### Example 17

The medium of any of Examples 15-16, wherein the instructions are further operable to configure the surgical visualization system to combine the sets of points received from the plurality of cameras using bundle adjustment.

### Example 18

The medium of any of Examples 15-17, wherein the instructions are further operable to configure the surgical visualization system to display a view of the interior of the cavity of the patient as viewed from a viewpoint of a virtual camera based on the three dimensional point cloud.

### Example 19

The medium of Example 18, wherein the instructions are further operable to configure the surgical visualization system to, based on receiving a command to modify the view of the interior of the cavity of the patient: (a) modify one or more of the virtual camera's position, focus and orientation; and (b) display an updated view of the interior of the cavity of the patient, wherein the updated view is of the interior of the cavity of the patient as viewed by the virtual camera after the modification.

### Example 20

The medium of Example 19, wherein the instructions are operable to configure the surgical visualization system to display the updated view of the interior of the cavity of the patient while holding each of the plurality of cameras stationary.

V. Miscellaneous

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they may be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that may penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A surgical visualization system comprising:
(a) a plurality of trocars, each trocar comprising a working channel;
(b) a plurality of cameras, wherein each camera from the plurality of cameras:
(i) has a corresponding trocar from the plurality of trocars;
(ii) is least partially inserted through the working channel of its corresponding trocar; and
(iii) is adapted to capture images of an interior of a cavity of a patient when inserted through the working channel of its corresponding trocar;
(c) a processor, wherein:
(i) for each camera from the plurality of cameras:
(A) the processor is in operative communication with that camera; and
(B) the processor is configured to receive a set of points corresponding to an image captured by that camera;
and
(ii) the processor is configured to generate a three dimensional point cloud representing the interior of the cavity of the patient based on combining the sets of points received from the plurality of cameras.

2. The surgical visualization system of claim 1, wherein the plurality of cameras comprises at least four cameras.

3. The surgical visualization system of claim 1, wherein the processor is configured to combine the sets of points received from the plurality of cameras using bundle adjustment.

4. The surgical visualization system of claim 1, wherein the processor is configured to display a view of the interior of the cavity of the patient as viewed from a viewpoint of a virtual camera based on the three dimensional point cloud.

5. The surgical visualization system of claim 4, wherein the processor is configured to, based on receiving a command to modify the view of the interior of the cavity of the patient:
(a) modifying one or more of the virtual camera's position, focus and orientation; and
(b) displaying an updated view of the interior of the cavity of the patient, wherein the updated view is of the interior of the cavity of the patient as viewed by the virtual camera after the modification.

6. The surgical visualization system of claim 5, wherein the processor is configured to display the updated view of the interior of the cavity of the patient while holding each of the plurality of cameras stationary.

7. The surgical visualization system of claim 1, wherein at least one camera from the plurality of cameras has a cross sectional area less than or equal to one square millimeter.

8. A method comprising:
(a) for each of a plurality of cameras inserting that camera at least partially through a corresponding trocar;
(b) using the plurality of cameras to capture images of an interior of a cavity of a patient; and
(c) a processor:
(i) receiving, from each camera from the plurality of cameras, a set of points corresponding to an image captured by that camera; and
(ii) generating a three dimensional point cloud representing the interior of the cavity of the patient based on combining the sets of points.

9. The method of claim 8, wherein the plurality of cameras comprises at least four cameras.

10. The method of claim 8, wherein the processor is configured to combine the sets of points received from the plurality of cameras using bundle adjustment.

11. The method of claim 8, wherein the processor is configured to display a view of the interior of the cavity of the patient as viewed from a viewpoint of a virtual camera based on the three dimensional point cloud.

12. The method of claim 11, wherein the processor is configured to, based on receiving a command to modify the view of the interior of the cavity of the patient:
(a) modify one or more of the virtual camera's position, focus and orientation; and
(b) display an updated view of the interior of the cavity of the patient, wherein the updated view is of the interior of the cavity of the patient as viewed by the virtual camera after the modification.

13. The method of claim 12, wherein the processor is configured to display the updated view of the interior of the cavity of the patient while holding each of the plurality of cameras stationary.

14. The method of claim 8, wherein each camera from the plurality of cameras has a cross sectional area less than or equal to one square millimeter.

15. A non-transitory computer readable medium storing instructions operable to configure a surgical visualization system to perform a set of steps comprising:
(a) capturing, using a plurality of cameras, a plurality of images of an interior of a cavity of a patient;
(b) receiving, from each camera from the plurality of cameras, a set of points corresponding to an image captured by that camera; and
(c) generating a three dimensional point cloud representing the interior of the cavity of the patient based on combining the sets of points.

16. The medium of claim 15, wherein the plurality of cameras comprises four cameras.

17. The medium of claim 15, wherein the instructions are further operable to configure the surgical visualization system to combine the sets of points received from the plurality of cameras using bundle adjustment.

18. The medium of claim 15, wherein the instructions are further operable to configure the surgical visualization system to display a view of the interior of the cavity of the patient as viewed from a viewpoint of a virtual camera based on the three dimensional point cloud.

19. The medium of claim 18, wherein the instructions are further operable to configure the surgical visualization system to, based on receiving a command to modify the view of the interior of the cavity of the patient:
(a) modify one or more of the virtual camera's position, focus and orientation; and
(b) display an updated view of the interior of the cavity of the patient, wherein the updated view is of the interior of the cavity of the patient as viewed by the virtual camera after the modification.

20. The medium of claim 19, wherein the instructions are operable to configure the surgical visualization system to display the updated view of the interior of the cavity of the patient while holding each of the plurality of cameras stationary.
